(19) United States
(12) Patent Application Publication
(10) Pub. No.: US 2022/0071880 A1
(43) Pub. Date: Mar. 10, 2022

(54) METHODS FOR TREATING CUTANEOUS AGING

(71) Applicant: Applied Therapeutics, Inc., New York, NY (US)

(72) Inventor: Shoshana SHENDELMAN, New York, NY (US)

(73) Assignee: Applied Therapeutics, Inc., New York, NY (US)

(21) Appl. No.: 17/428,917

(22) PCT Filed: Feb. 12, 2020
(86) PCT No.: PCT/US2020/017913

§ 371 Date: Aug. 5, 2021

Related U.S. Application Data
(60) Provisional application No. 62/804,413, filed on Feb. 12, 2019.

(51) Int. Cl. A61K 8/49; A61Q 19/08

(52) U.S. Cl. CPC A61K 8/494; A61K 2800/782; A61Q 19/08; A61K 8/4933

(57) ABSTRACT

The disclosure relates to methods for treating cutaneous aging by administering to a subject in need thereof a therapeutically effective amount of an aldose reductase inhibitor.

### RELATED APPLICATION

This patent application claims the benefit of U.S. Provisional Patent Application No. 62/804,413 filed on Feb. 12, 2019, the entire disclosure of which is incorporated herein by reference.

### BACKGROUND

Cutaneous aging is a complex phenomenon affecting various layers of the skin, but the major cellular and extracellular matrix changes are seen in the dermis. Normal human dermis consists primarily of an extracellular matrix that contains collagen, elastin and glycosaminoglycans/proteoglycans (e.g., hyaluronic acid) and three major types of cells: fibroblasts, macrophages and adipocytes. The cutaneous aging process involves two distinct processes, the intrinsic aging process and the extrinsic aging processes. The intrinsic aging process is the slow degenerative aging process that is primarily influenced by individual genetic and epigenetic characteristics. The extrinsic aging process is influenced by a variety of environmental factors, such as exposure to ultraviolet light, pollution and lifestyle, with exposure to ultraviolet light recognized as a primary cause of extrinsic cutaneous aging. Uitto J and Berstein E F, J Investigative Dermatology Symposium Proceedings 3:41-44 1998. The extrinsic aging process can amplify and accelerate the intrinsic aging process. Crisan M et al., Plos One, 8:10: e75003. doi:10.1371/journal.pone.0075003.

Cutaneous aging is characterized by a number of well-characterized changes in the skin, such as, thinning of the epidermis, reduction in the number of melanocytes, changes in elasticity (i.e., elastosis) which can produce a leathery appearance, blood vessels in the dermis become more fragile leading to bruising or bleeding under the skin, reduced production of oil by sebaceous glands and other changes. These and other changes result in the characteristic formation of wrinkles, lines, creases in the skin, and sagging in the skin as it ages, and also to decreased sensation (e.g., of touch, pressure, vibration, heat and cold), changes in pigmentation, increased susceptibility to injury, and decreased healing ability.

Advanced glycation end products (AGEs) are produced in the body through the nonenzymatic glycation and oxidation of proteins and lipids. Ramasamy R et al. Glycobiology, 13:7, 16R-28R, 2005. AGEs are produced naturally in the body through a process that also produces reactive oxygen species (ROS) leading to oxidative stress. AGEs bind to and activate the receptor of advanced glycation end products (RAGE), leading to a number of changes that are associated with aging, such as further increased production of reactive oxygen species (ROS) and oxidative stress, modulated expression of proinflammatory and prothrombotic molecules including IL-2, V-CAM1 and other cellular adhesion molecules, modulated production of collagen by fibroblasts, and matrix modifying proteins in smooth muscle cells. Id. at 17R.

The polyol pathway also contributes to non-enzymatic glycation and the production of AGEs. In this pathway, aldose reductase (AR) reduces glucose to sorbitol which is then converted to fructose by the action of sorbitol dehydrogenase. The conversion of sorbitol to fructose involves the production of NADH, which potentially leads to increased production of ROS through the action of NADH oxidase. Tang et al., Frontiers in Pharmacology, vol. 3, article 87, 2012. doi:10.3389/fphar.2012.00087. The fructose is converted to fructose-3-phosphate through the action of 3-phosphokinase and 3-deoxyglucasone (3-DG) is produced as a product of this reaction. 3-DG is a major precursor in the generation of a variety of AGEs including carboxymethyl lysine (CML) adducts (AGE-CML). Glycation and AGEs can also induce the activity of aldose reductase (AR), which is the rate limiting enzyme in the polyol pathway, further exacerbating the damage and increasing production of AGEs. Ramasamy R et al. at 18R.

The role of the polyol pathway, 3-DG, AGEs, ROS and oxidative stress in cardiovascular, renal and neuronal complications of type 2 diabetes has been the subject of investigation. Id. Systemic inhibition of AR in patients with type 2 diabetes using the AR inhibitor epalrestat reduced plasma levels of AGEs. Id. These results are recognized as supporting the premise that elevated 3-DG levels contribute to diabetic complications, and the observations that type 2 diabetic patients with higher 3-DG levels experience more severe complications of type 2 diabetes. Id. But it is also recognized that fructose produced by the polyol pathway might directly induce protein oxidation and AGE formation in type 2 diabetes.

Non-enzymatic glycation and oxidative damage is associated with cutaneous aging and affects cells and structural proteins in the skin, such as collagen, elastin and glycosaminoglycans. Glycation and oxidative damage to the extracellular matrix of the dermis affects growth, differentiation and motility of fibroblasts, the cytokine response, enzymatic activity and vascular homeostasis of the skin. Non-enzymatic glycation and AGEs play a role in both the intrinsic and extrinsic cutaneous aging processes. Crisan M et al. The functional and structural changes, including advanced glycation, associated with cutaneous aging are seen in the dermis before age 35 and increase rapidly thereafter. Id. at 4. The rate of increase in the functional and structural changes of cutaneous aging is amplified by the extrinsic aging process (such as exposure to UV light). Id. Skin aging has been shown to result in oxidative damage, and exposure to UV light further accelerates the aging process in part by increasing oxidative damage (Starr et al., “Chapter 2-Sking Aging and Oxidative Stress”, in Aging: Oxidative Stress and Dietary Antioxidants, V. Preedy ed., 2014, pages 15-22, Academic Press; doi.org/10.1016/B978-0-12-405933-7.00002-0).

There is a need for new methods for treating cutaneous aging.

### SUMMARY

This disclosure relates to methods for treating cutaneous aging by administering a therapeutically effective amount of an AR inhibitor to a subject in need thereof. Preferably, the AR inhibitor is administered topically to the skin. Without wishing to be bound by any particular theory, it is believed that inhibition of AR in the skin can reduce or prevent the formation of AGEs in the skin, as well as down-stream mediators that contribute to cellular and structural changes of cutaneous aging, such as ROS and collagen cross-linking. Accordingly, the methods disclosed herein can reduce or delay the signs of cutaneous aging, such as the appearance of as lines, creases, wrinkles and crepey skin and loss of elasticity or firmness of the skin. In the practice of the disclosed methods, the aldose reductase inhibitor can be topically administered to the skin, for example by application to the surface of the skin (e.g., of a topical formulation that contains the aldose reductase inhibitor). The aldose reductase inhibitor can be applied to the surface of any desired area of the skin. For example, the aldose reductase inhibitor can be applied to the surface of skin that is typically exposed in social settings, such as the skin of the face, neck, chest, arms, hands or any combination of the foregoing, to reduce or delay cutaneous aging in those areas of the skin.

In the practice of the methods disclosed herein, the aldose reductase inhibitor can be administered in an amount effective to reduce AGE formation in the skin, for example, due to intrinsic processes or extrinsic causes such as exposure to ultraviolet light. In the practice of the methods disclosed herein, the aldose reductase inhibitor can be administered in an amount effective to reduce ROS in the skin. In the practice of the methods disclosed herein, the aldose reductase inhibitor can be administered in an amount effective to reduce 3-GC in the skin. In the practice of the methods disclosed herein, the aldose reductase inhibitor can be administered in an amount effective to reduce sorbitol or fructose in the skin. In the practice of the methods disclosed herein, the aldose reductase inhibitor can be administered in an amount effective to reduce oxidative damage in the skin. In the practice of the methods disclosed herein, the aldose reductase inhibitor can be administered in an amount effective to reduce or delay fragmentation, break down and/or cross-linking of extracellular matrix proteins in the skin. In the practice of the methods disclosed herein, the aldose reductase inhibitor can be administered in an amount effective amount is effective to reduce fragmentation, break down and/or cross-linking of collagen and/or elastin in the dermis.

This disclosure also relates to a method of reducing advanced glycation end products (AGEs) in the skin of a subject, comprising topically administering to the skin of the subject a therapeutically effective amount of an aldose reductase inhibitor. If desired, the method can further comprise systemically administering the same or different AR inhibitor to the subject, preferably by oral administration.

This disclosure also relates to a method of reducing reactive oxygen species in the skin of a subject, comprising topically administering to the skin of the subject a therapeutically effective amount of an aldose reductase inhibitor. If desired, the method can further comprise systemically administering the same or different AR inhibitor to the subject, preferably by oral administration.

This disclosure also relates to a method of reducing 3-GC in the skin of a subject, comprising topically administering to the skin of the subject a therapeutically effective amount of an aldose reductase inhibitor. If desired, the method can further comprise systemically administering the same or different AR inhibitor to the subject, preferably by oral administration.

This disclosure also relates to a method of reducing fructose in the skin of a subject, comprising administering topically administering to the skin of the subject a therapeutically effective amount of an aldose reductase inhibitor. If desired, the method can further comprise systemically administering the same or different AR inhibitor to the subject, preferably by oral administration.

This disclosure also relates to a method of reducing reduce oxidative damage in the skin of a subject, comprising topically administering to the skin of the subject a therapeutically effective amount of an aldose reductase inhibitor. If desired, the method can further comprise systemically administering the same or different AR inhibitor to the subject, preferably by oral administration.

This disclosure also relates to a method of reducing or delaying fragmentation, break down and/or cross-linking of extracellular matrix proteins in the skin of a subject, comprising topically administering to the skin of the subject a therapeutically effective amount of an aldose reductase inhibitor. The extracellular matrix protein can be collagen (e.g., collagen in the dermis), elastin (e.g., elastin in the dermis) or other ECM protein. If desired, the method can further comprise systemically administering the same or different AR inhibitor to the subject, preferably by oral administration.

This disclosure also relates to use of an aldose reductase inhibitor for treatment of a disease or condition described herein. This disclosure also relates to an aldose reductase inhibitor for use in the manufacture of a medicament for treatment of a disease or condition described herein. This disclosure also relates to a pharmaceutical composition for treatment of a disease or condition described herein that comprises an aldose reductase inhibitor as an active agent.

Typically, the inhibitor of aldose reductase is administered at least once a day in the practice of the methods disclosed herein.

### BRIEF DESCRIPTION OF THE DRAWINGS

FIGS. 1A and 1B are fluorescence images of cultured human skin (keratinocytes) that were exposed to H₂O₂ to cause oxidative stress. The cells were analyzied for cytosolic reactive oxygen species by staining with dihydroethidium (DHE). FIG. 1A depicts the skin cell damage in the presence of H₂O₂ as assessed by DHE staining, and the presence of cytosolic reactive oxygen species is shown by fluorescence. FIG. 1B shows that Compound A attenuated DHE fluorescence, demonstrating that Compound A attenuated the formation of cytosolic reactive oxygen species.

FIGS. 2A and 2B are fluorescence images of cultured human skin (keratinocytes) that were exposed to H₂O₂ to cause oxidative stress. The cells were analyzied for mitochondrial stress by staining with Mitosox. FIG. 2A depicts the skin cell damage in the presence of H₂O₂ as assessed by Mitosox staining. FIG. 2B shows that Compound A reduced Mitisox fluorescence, demonstrating that Compound A attenuated mitochondrial oxidative stress.

### DETAILED DESCRIPTION

This disclosure relates to the use of AR inhibitors for the treatment of cutaneous aging. The inventor has discovered that inhibition of AR in the skin can reduce the levels of AGEs, ROS and oxidative stress in the skin, and reduce or delay cellular and structural changes in the skin, such as fragmentation and cross-lining of collagen, that contribute to aging of the skin. Accordingly, the methods described herein, can be used to reduce or delay the appearance of signs of cutaneous aging, such as lines, creases, wrinkles, decreased firmness or elasticity and crepey skin. When desirable, the AR inhibitor can be applied topically to the skin to treat cutaneous aging, avoiding systemic effects.

Where a range of values is provided in this disclosure, it is intended that each intervening value between the upper and lower limit of that range and any other stated or intervening value in that stated range is encompassed within the disclosure. For example, if a range of 1 μM to 8 μM is stated, it is intended that 2 μM, 3 μM, 4 μM, 5 μM, 6 μM, and 7 μM are also explicitly disclosed, as well as the range of values greater than or equal to 1 μM and the range of values less than or equal to 8 μM.

The singular forms “a,” “an,” and “the” include plural referents unless the context clearly dictates otherwise. Thus, for example, reference to a “compound of Formula I” includes a single compound as well as two or more of the same or different compounds; reference to an “excipient” includes a single excipient as well as two or more of the same or different excipients, and the like.

The word “about” means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

A “subject” can be any animal, particularly a mammal, and including, but not limited to, humans, domestic animals, such as feline or canine subjects, farm animals, such as but not limited to bovine, equine, caprine, ovine, avian and porcine subjects, wild animals (whether in the wild or in a zoological garden), research or laboratory animals, such as mice, rats, rabbits, goats, sheep, pigs, dogs, cats, etc., avian species, such as chickens, turkeys, songbirds, and the like. Preferably, the “subject” is a human.

In order to provide a complete, concise and clear description of the various embodiments, this disclosure includes descriptions of various components, groups of components, ranges and other elements of the broader disclosure. It is intended that such elements can be variously combined to provide additional embodiments of the disclosure. It is also intended that any disclosed features (e.g., substituent, analog, compound, structure, component) including individual members of any disclosed group, including any sub-ranges or combinations of sub-ranges within the group, may be excluded from the disclosure or any embodiments of the disclosure for any reason.

### I. Methods

This disclosure relates to methods for the treatment of cutaneous aging, comprising administering to a subject in need thereof a therapeutically effective amount of a compound that inhibits aldose reductase activity. Accordingly, the methods described herein, can be used to reduce or delay the appearance of signs of cutaneous aging, such as lines, creases, wrinkles and crepey skin. When desirable, the AR inhibitor can be applied topically to the skin to treat cutaneous aging, avoiding systemic effects. For example, the AR inhibitor can be topically applied to the surface of the skin, such as the skin of the face, neck, chest, hands or other desired area of the body.

As used herein, the term “treating” refers to therapy to reduce or delay the appearance of a sign or characteristic of cutaneous aging. For example, reducing, arresting or delaying the signs, characteristics and/or underlying structural changes that are characteristic of cutaneous aging, such as, thinning of the epidermis, reduction in the number of melanocytes, changes in elasticity (i.e., elastosis), blood vessels in the dermis becoming more fragile leading to bruising or bleeding under the skin, reduced production of oil by sebaceous glands and structural changes in the extracellular matrix, such as, fragmentation, break down and/or cross-linking of collagen and elastin in the dermis. These and other changes result in the characteristic formation of wrinkles, lines, crease and sagging in the skin as it ages, and also to decreased sensation (e.g., of touch, pressure, vibration, heat and cold), changes in pigmentation, increased susceptibility to injury, and decreased healing ability.

As used herein “a therapeutically effective amount” is an amount of a compound that is sufficient to achieve the desired therapeutic effect under the conditions of administration, such as an amount that reduces or delays the appearance of a sign or characteristic of cutaneous aging.

A therapeutically effective amount can be an amount that inhibits AR in skin cells, for example in cells in the dermis. In some examples, the therapeutically effective amount is an amount sufficient to reduce intracellular aldose reductase activity at least by about 5%, at least about 10%, at least about 20%, at least about 30%, at least about 40%, at least about 50%, at least about 60%, at least about 70%, at least about 80%, at least about 90%, at least about 95%, at least about 99%, or more, e.g., about 100% (e.g., compared to pre-treatment level). A therapeutically effective amount can be an amount sufficient to reduce AGEs (e.g., AGE-CML) in skin, to reduce fructose in skin, and/or to reduce 3-DG in skin. A therapeutically effective amount can reduce or delay the onset or accumulation of changes in the extracellular matrix (ECM) of the skin, particularly in the dermis. For example, a therapeutically effective amount can reduce or delay accumulation of fragmented and/or cross-linked ECM proteins in the dermis, such as collagen and elastin. A therapeutically effective amount can also reduce or delay the signs of cutaneous aging, such as the appearance of fine lines, wrinkles, creases, crepey skin, loss of skin tone (e.g., sagging), changes in pigmentation (e.g, age spots) and the like. The actual amount administered can be determined by an ordinarily skilled clinician based upon, for example, the subjects age, weight, sex, general heath and tolerance to drugs, severity of disease, dosage form selected, route of administration and other factors. The amount of an AR inhibitor that is administered systemically can be from about 0.5 to about 60 mg/kg body weight per day, such as from about 1.0 to 10 mg/kg, and typically a lower amount is administered topically, e.g., by applying a suitable topical formulation or composition (e.g., a gel, foam, cream, serum or solution) containing the AR inhibitor directly to desired areas of the skin (e.g., face, neck, chest, hands) one or more times per day.

The methods and treatment regimens described in this disclosure can reduce AGEs (e.g., AGE-CML) in skin, reduce fructose in skin, reduce 3-DG in skin and/or reduce or delay fragmentation, break down and/or cross-linking of extracellular matrix proteins (e.g., collagen, elastin) in the skin by at least about 10%, at least about 20%, at least about 30%, at least about 40%, at least about 50%, at least about 60%, at least about 70%, at least about 80%, at least about 90%, at least about 95%, at least about 99%, or more, e.g., about 100%, compared to pre-treatment level or to levels that are typical and characteristic of a patient of the same age and skin type (e.g., the same Fitzpatric skin type or phototype).

This disclosure also relates to a method of reducing advanced glycation end products (AGEs) in the skin of a subject, comprising topically administering to the skin of the subject a therapeutically effective amount of an aldose reductase inhibitor. If desired, the method can further comprise systemically administering the same or different AR inhibitor to the subject, preferably by oral administration.

This disclosure also relates to a method of reducing reactive oxygen species in the skin of a subject, comprising topically administering to the skin of the subject a therapeutically effective amount of an aldose reductase inhibitor. If desired, the method can further comprise systemically administering the same or different AR inhibitor to the subject, preferably by oral administration.

This disclosure also relates to a method of reducing 3-GC in the skin of a subject, comprising topically administering to the skin of the subject a therapeutically effective amount of an aldose reductase inhibitor. If desired, the method can further comprise systemically administering the same or different AR inhibitor to the subject, preferably by oral administration.

This disclosure also relates to a method of reducing fructose in the skin of a subject, comprising administering topically administering to the skin of the subject a therapeutically effective amount of an aldose reductase inhibitor. If desired, the method can further comprise systemically administering the same or different AR inhibitor to the subject, preferably by oral administration.

This disclosure also relates to a method of reducing reduce oxidative damage in the skin of a subject, comprising topically administering to the skin of the subject a therapeutically effective amount of an aldose reductase inhibitor. If desired, the method can further comprise systemically administering the same or different AR inhibitor to the subject, preferably by oral administration.

This disclosure also relates to a method of reducing or delaying fragmentation, break down and/or cross-linking of extracellular matrix proteins in the skin of a subject, comprising topically administering to the skin of the subject a therapeutically effective amount of an aldose reductase inhibitor. The extracellular matrix protein can be collagen (e.g., collagen in the dermis), elastin (e.g., elastin in the dermis) or other ECM protein. If desired, the method can further comprise systemically administering the same or different AR inhibitor to the subject, preferably by oral administration.

The AR inhibitor administered in accordance with the disclosed methods can be any compound that inhibits AR activity, such as a small molecule compound (e.g., having a size of 5 kDa or less), a biologic agent (e.g., an inhibitory RNA directed against aldose reductase) or a combination thereof.

In one example, the disclosed methods comprise administering to a subject in need thereof a therapeutically effective amount of zopolrestat.

In another example, the disclosed methods comprise administering to a subject in need thereof a therapeutically effective amount of epalrestat.

In other examples, the disclosed methods comprises administering to a subject in need thereof a therapeutically effective amount of an aldose reductase inhibitor, that is not ponalrestat, epalrestat, sorbinil or sorbinol, imirestat, AND-138, CT-112, zopolrestat, zenarestat, BAL-AR18, AD-5467, M-79175, tolrestat, alconil, statil, berberine or SPR-210.

In some preferred aspects, the disclosed methods comprise administering to a subject in need thereof a therapeutically effective amount of a compound of any one of Formulas I-VI. In other preferred aspects, the disclosed methods comprise administering to a subject in need thereof a therapeutically effective amount of a compound of Formulas II or Formula III.

The methods disclosed herein can be practiced by administering a single dosage or single administration (e.g., as a single injection or deposition) of one or more AR inhibitors, but typically involve an administration regimen under which an AR inhibitor is administered at least once daily for the desired course of treatment. For example, the AR inhibitor can be administered once daily, twice daily, three times daily, four times daily or more to a subject in need thereof for a period of from about 2 to about 28 days, or from about 7 to about 10 days, or from about 7 to about 15 days, or longer. The disclosed methods include long-term administration regimens, for example with the administration of an AR inhibitor at least once daily for a period of weeks, months, years or decades.

### II. AR Inhibitors

Suitable small molecule AR inhibitors are known in the art and are disclosed herein. Small molecule AR inhibitors include ponalrestat, sorbinil, sorbinol, imirestat, AND-138, CT-112, zenarestat, BAL-AR18, AD-5467, M-79175, tolrestat, alconil, statil, berberine, SPR-210 zopolrestat, epalrestat, the compounds disclosed in U.S. Pat. Nos. 8,916,563, 9,650,383, 10,150,779 and the compounds disclosed herein. Preferred AR inhibitors for use in the invention include zopolrestat, epalrestat, the compounds disclosed in U.S. Pat. Nos. 8,916,563, 9,650,383, 10,150,779 and the compounds disclosed herein. The AR inhibitors can be administered in any suitable molecular form including pharmaceutically acceptable salts, solvates, prodrugs, and compounds that contain stable isotopic forms of one or more atoms, e.g., deuterium in place of hydrogen.

### AR Inhibitors of Formulas I and II

In one example, the AR inhibitor is a compound of Formula (I) or pharmaceutically acceptable salts, prodrugs and solvates thereof,

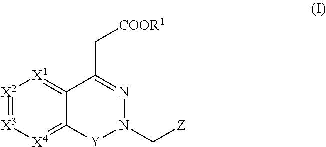

wherein,

R¹ is H, (C₁-C₆)-alkyl, (C₁-C₆)-hydroxyalkyl, or (C₁-C₆)-aminoalkyl;

X¹ is N or CR³;

X² is N or CR⁴;

X³ is N or CR⁵;

X⁴ is N or CR⁶; with the proviso that two or three of X¹, X², X³, or X⁴ are N;

Y is a bond, C═O, C═S, C═NH, or C═N(C₁-C₄)-alkyl;

Z is

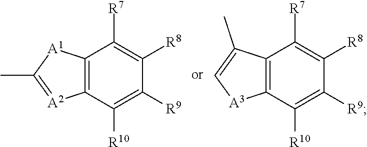

A¹ is NR¹¹, O, S or CH₂;

A² is N or CH;

A³ is NR¹¹, O, or S;

R³ through R¹⁰ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl; or two of R³ through R⁶ or two of R⁷ through R¹⁰ taken together are (C₁-C₄)-alkylenedioxy; and

R¹¹ is hydrogen, C₁-C₄ alkyl, or C(O)O—(C₁-C₄)-alkyl.

It will be recognized by those of skill in the art that the designation of Z is

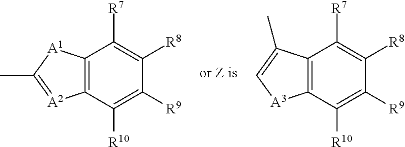

indicates that when Z is

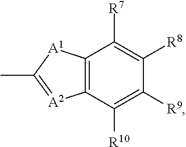

the compounds of formula (I) encompass

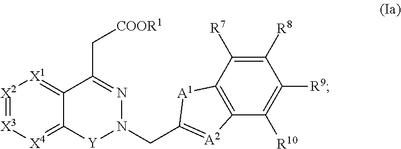

and when Z is

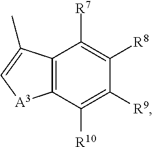

the compounds of formula (I) encompass

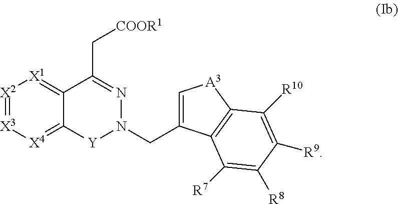

In certain embodiments, R¹ is hydrogen or (C₁-C₆)-alkyl. In certain embodiments, R¹ is hydrogen. In certain embodiments, R¹ is (C₁-C₆)-alkyl. In certain embodiments, R¹ is tert-butyl.

In certain embodiments, R³ through R¹⁰ are independently hydrogen, halogen or haloalkyl. In certain embodiments, R³ through R¹⁰ are independently hydrogen, halogen or trihaloalkyl.

In certain embodiments, R³ through R⁶ are hydrogen.

In certain embodiments, R⁷ through R¹⁰ are independently hydrogen, halogen or haloalkyl. In certain embodiments, R⁷ through R¹⁰ are independently hydrogen, halogen or trihaloalkyl.

In certain embodiments, R⁷ and R¹⁰ are hydrogen.

In certain embodiments, R⁸ is hydrogen, halogen or haloalkyl. In certain embodiments, R⁸ is hydrogen. In certain embodiments, R⁸ is halogen. In certain embodiments, R⁸ is haloalkyl.

In certain embodiments, R⁹ is hydrogen, halogen or haloalkyl. In certain embodiments, R⁹ is hydrogen. In certain embodiments, R⁹ is halogen. In certain embodiments, R⁹ is haloalkyl.

In certain embodiments, Y is C═O, C═S, C═NH, or C═N(C₁-C₄)-alkyl. In certain embodiments, Y is C═O or C═S. In certain embodiments, Y is C═O. In certain embodiments, Y is C═S. In certain embodiments, Y is C═NH, or C═N(C₁-C₄)-alkyl.

In certain embodiments, A¹ is NR¹¹, S or CH₂. In certain embodiments, A¹ is NR¹¹ or O. In certain embodiments, A¹ is NR¹¹ or S. In certain embodiments, A¹ is NR¹¹. In certain embodiments, A¹ is O. In certain embodiments, A¹ is S.

In certain embodiments, A² is N or CH. In certain embodiments, A¹ is N. In certain embodiments, A¹ is CH.

In certain embodiments, A³ is O or S. In certain embodiments, A³ is O. In certain embodiments, A³ is S.

In certain embodiments, X¹ and X⁴ are nitrogen.

In certain embodiments, X¹ and X² are nitrogen.

In certain embodiments, X¹ and X³ are nitrogen.

In certain embodiments, X² and X³ are nitrogen.

In certain embodiments, X² and X⁴ are nitrogen.

In certain embodiments, X³ and X⁴ are nitrogen.

In certain embodiments, Z is

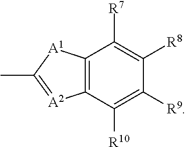

In certain embodiments, Z is

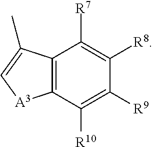

In certain embodiments, R¹ is hydrogen or (C₁-C₆)-alkyl;

X¹ and X⁴ are N;

X² is CR⁴;

X³ is CR⁵;

Y is C═O;

Z is

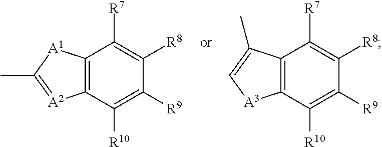

A¹ is NR¹¹, O, or S;

A² is N;

A³ is O, or S;

R⁴ and R⁵ are hydrogen;

R⁷ through R¹⁰ are independently hydrogen, halogen, cyano, acyl, haloalkyl, haloalkoxy, haloalkylthio, (C₁-C₄)-alkyl, (C₁-C₄)-alkoxy, (C₁-C₄)-alkylthio, (C₁-C₄)-alkylsulfinyl, or (C₁-C₄)-alkylsulfonyl; and

R¹¹ is hydrogen, C₁-C₄ alkyl, or C(O)O(C₁-C₄)-alkyl.

In certain embodiments, R¹ is hydrogen or tert-butyl;

X¹ and X⁴ are N;

X² is CR⁴;

X³ is CR⁵;

Y is C═O;

Z is

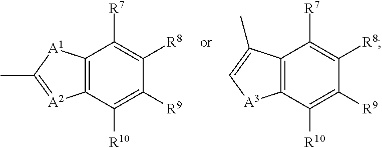

A¹ is NR¹¹, O or S;

A² is N;

A³ is O or S;

R⁴ and R⁵ are hydrogen;

R⁷ through R¹⁰ are independently hydrogen, halogen, or haloalkyl; and

R¹¹ is hydrogen, (C₁-C₄)-alkyl, or C(O)O-tert-butyl.

In certain embodiments, R¹ is hydrogen or tert-butyl;

X¹ and X⁴ are N;

X² is CH;

X³ is CH;

Y is C═O;

Z is

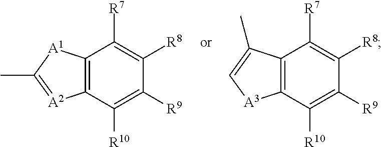

A¹ is NR¹¹, O or S;

A² is N;

A³ is O or S;

R⁷, R⁸ and R¹⁰ are independently hydrogen, halogen, or haloalkyl;

R⁹ is halogen, or haloalkyl; and

R¹¹ is hydrogen or methyl.

In certain embodiments, R¹ is hydrogen or tert-butyl;

X¹ and X⁴ are N;

X² is CH;

X³ is CH;

Y is C═O;

Z is

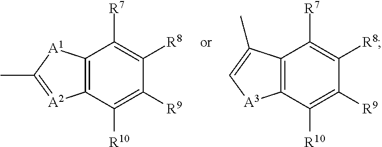

A¹ is NR¹¹, O or S;

A² is N;

A³ is O or S;

R⁷, R⁸ and R¹⁰ are independently hydrogen, halogen, or haloalkyl;

R⁹ is chlorine, or trifluoromethyl; and

R¹¹ is hydrogen or methyl.

In certain embodiments, the AR inhibitor is a compound of Formula (II) or pharmaceutically acceptable salt or solvate thereof:

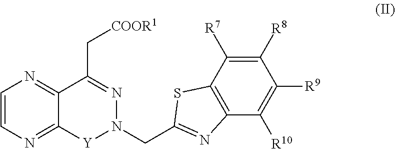

Wherein R¹, R⁷-R⁹ and Y are as described in Formula I, and preferable wherein R¹ is hydrogen or (C₁-C₆)-alkyl and Y is C═O. Exemplary compounds of Formula II include the following and salts thereof:

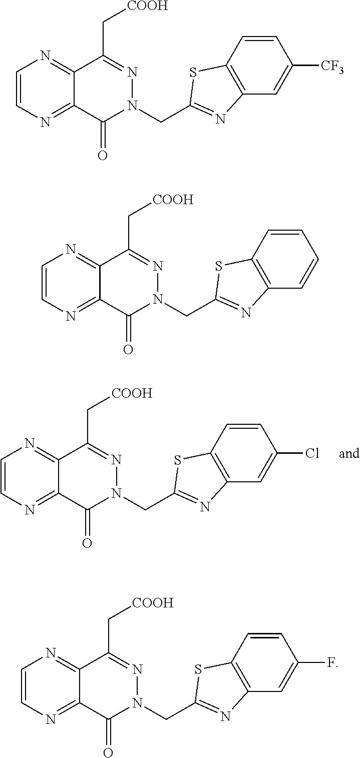

Compounds of Formula III

The AR inhibitors can be a compound of Formula (III) or pharmaceutically acceptable salts, pro-drugs and solvates thereof,

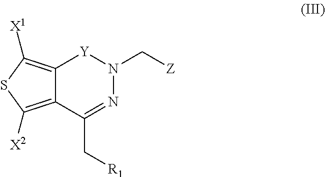

wherein,
Rn is CO₂R² or CO₂⁻X⁺;
R² is H, (C₁-C₆)-alkyl, (C₁-C₆)-hydroxyalkyl, or (C₁-C₆)-aminoalkyl;
X¹ is H or halogen;
X² is H or halogen;
Y is a bond, C═O, C═S, C═NH, or C═N(C₁-C₄)-alkyl;
Z is

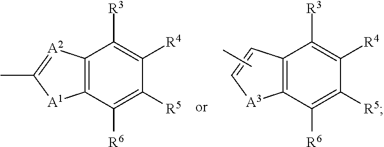

A¹ is NR⁷, O, S or CH₂;
A² is N or CH;
A³ is NR⁷, O, or S;
R³ through R⁶ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl;
R⁷ is hydrogen, C₁-C₄ alkyl, or C(O)O—(C₁-C₄)-alkyl; and
X⁺ is a counter ion.
It will be recognized by those of skill in the art that the designation of

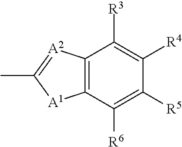

### or Z is

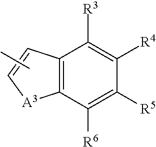

indicates that when Z is

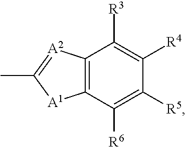

the compounds of Formula (III) are understood to encompass

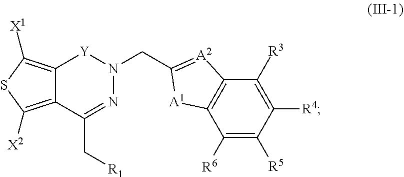

and when Z is

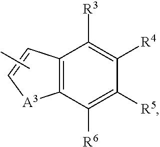

the compounds of Formula (I) are understood to encompass

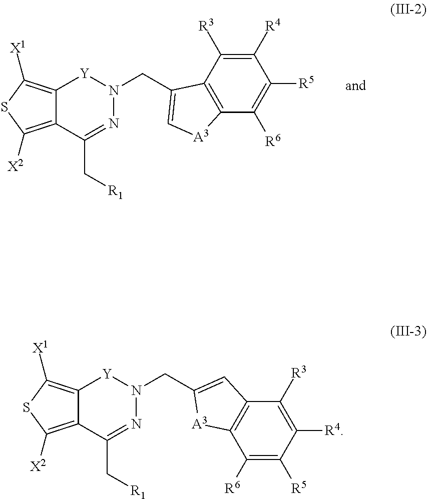

In certain embodiments, R¹ is CO₂R² or CO₂X⁺. In certain embodiments, R¹ is CO₂R². In certain embodiments, R¹ is CO₂X⁺.

In certain embodiments, R² is hydrogen or (C₁-C₆)-alkyl. In certain embodiments, R² is hydrogen or (C₁-C₄)-alkyl. In certain embodiments, R² is hydrogen or (C₁-C₃)-alkyl. In certain embodiments, R² is hydrogen, methyl, or ethyl. In certain embodiments, R² is hydrogen or methyl. In certain embodiments, R² is methyl or ethyl. In certain embodiments, R² is methyl. In certain embodiments, R² is hydrogen. In certain embodiments, R² is (C₁-C₆)-alkyl. In certain embodiments, R² is (C₁-C₆)-n-alkyl. In certain embodiments, R² is (C₁-C₂)-alkyl. In certain embodiments, R² is (C₁-C₃)-alkyl. In certain embodiments, R² is (C₁-C₄)-alkyl. In certain embodiments, R² is tert-butyl.

In certain embodiments, R³ through R⁶ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl.

In certain embodiments, R³ through R⁶ are independently hydrogen, halogen or haloalkyl. In certain embodiments, R³ through R⁶ are independently hydrogen, halogen or trihaloalkyl.

In certain embodiments, R³ and R⁶ are hydrogen. In certain embodiments, R³, R⁵, and R⁶ are hydrogen.

In certain embodiments, R⁴ is hydrogen, halogen or haloalkyl. In certain embodiments, R⁴ is hydrogen. In certain embodiments, R⁴ is halogen. In certain embodiments, R⁴ is haloalkyl. In certain embodiments, R⁴ is CF₃.

In certain embodiments, R³ through R⁶ are hydrogen. In certain embodiments, R³, R⁵, R⁶ are hydrogen and R⁴ is halogen or haloalkyl. In certain embodiments, R³, R⁵, R⁶ are hydrogen and R⁴ is haloalkyl. In certain embodiments, R³, R⁵, R⁶ are hydrogen and R⁴ is CF₃. In certain embodiments, R³, R⁵, R⁶ are hydrogen and R⁴ is halogen. In certain embodiments, R³, R⁵, R⁶ are hydrogen and R⁴ is F. In certain embodiments, R³, R⁵, R⁶ are hydrogen and R⁴ is Cl.

In certain embodiments, Y is C═O, C═S, C═NH, or C═N(C₁-C₄)-alkyl. In certain embodiments, Y is C═O or C═S. In certain embodiments, Y is C═O. In certain embodiments, Y is C═S. In certain embodiments, Y is C═NH, or C═N(C₁-C₄)-alkyl.

In certain embodiments, A¹ is NR⁷, O, S or CH₂. In certain embodiments, A¹ is NR⁷, O, or S. In certain embodiments, A¹ is NR⁷, S or CH₂. In certain embodiments, A¹ is NR⁷ or O. In certain embodiments, A¹ is NR⁷ or S. In certain embodiments, A¹ is NR⁷. In certain embodiments, A¹ is O. In certain embodiments, A¹ is S.

In certain embodiments, A² is N or CH. In certain embodiments, A² is N. In certain embodiments, A² is CH.

In certain embodiments, A³ is NR⁷, O, or S. In certain embodiments, A³ is O. In certain embodiments, A³ is S. In certain embodiments, A³ is NR⁷.

In certain embodiments, X¹ and X² are hydrogen.

In certain embodiments, X¹ and X² are halogen. In certain embodiments, X¹ and X² are Cl.

In certain embodiments, X¹ and X² are independently hydrogen or halogen. In certain embodiments, X¹ is hydrogen and X² is Cl. In certain embodiments, X¹ is Cl and X² is hydrogen.

In certain embodiments, Z is

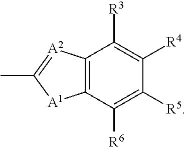

In certain embodiments, Z is

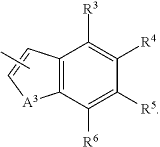

In certain embodiments, R⁷ is hydrogen, C₁-C₄ alkyl, or C(O)O—(C₁-C₄)-alkyl. In certain embodiments, R⁷ is hydrogen. In certain embodiments, R⁷ is C₁-C₄ alkyl. In certain embodiments, R⁷ is C₁-C₃ alkyl. In certain embodiments, R⁷ is C₁-C₂ alkyl. In certain embodiments, R⁷ is C₁-C₄ n-alkyl. In certain embodiments, R⁷ is C₁-C₃ n-alkyl. In certain embodiments, R⁷ is C(O)O—(C₁-C₄)-alkyl. In certain embodiments, R⁷ is C(O)O—(C₁-C₃)-alkyl. In certain embodiments, R⁷ is C(O)O—(C₁-C₂)-alkyl. In certain embodiments, R⁷ is C(O)O—(C₁-C₄)-n-alkyl. In certain embodiments, R⁷ is C(O)O—(C₁-C₃)-n-alkyl.

In certain embodiments, R¹ is CO₂R²;
R² is H or (C₁-C₆)-alkyl;
X¹ is H;
X² is H;
Y is C═O;
Z is

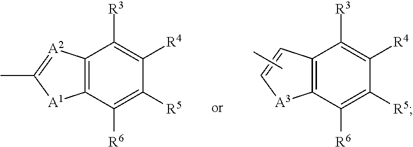

A¹ is NR⁷, O, or S;
A² is N;
A³ is O or S;
R³ through R⁶ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl; and
R⁷ is hydrogen, C₁-C₄ alkyl, or C(O)O—(C₁-C₄)-alkyl.

In certain embodiments, R¹ is CO₂R²;
R² is H or tert-butyl;
X¹ is H;
X² is H;
Y is C═O;
Z is

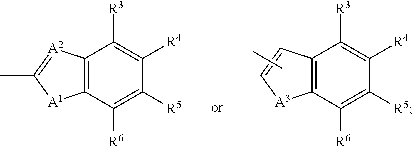

A¹ is NR⁷, O, or S;
A² is N;
A³ is O or S;
R⁶ through R⁶ are independently hydrogen, halogen, haloalkyl; and
R⁷ is hydrogen, C₁-C₄ alkyl, or C(O)O—(C₁-C₄)-alkyl.
In certain embodiments, R¹ is CO₂R²;
R² is H or tert-butyl;
X¹ is H;
X² is H;
Y is C═O;
Z is

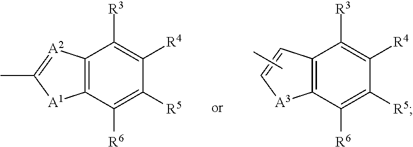

A¹ is NR⁷, O, or S;
A² is N;
A³ is O or S;
R³, R⁵, and R⁶ are hydrogen;
R⁴ is hydrogen, halogen, or haloalkyl; and
R⁷ is hydrogen, C₁-C₄ alkyl, or C(O)O—(C₁-C₄)-alkyl.
In certain embodiments, R¹ is CO₂R²;
R² is H or (C₁-C₆)-alkyl;
X¹ is halogen;
X² is halogen;
Y is C═O;
Z is

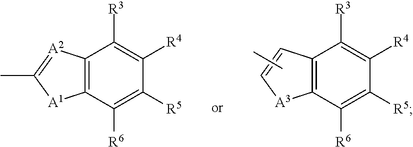

A¹ is NR⁷, O, or S;
A² is N;
A³ is O or S;
R³ through R⁶ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl; and
R⁷ is hydrogen, C₁-C₄ alkyl, or C(O)O—(C₁-C₄)-alkyl.
In certain embodiments, R¹ is CO₂R²;
R² is H or tert-butyl;
X¹ is halogen;
X² is halogen;
Y is C═O;
Z is

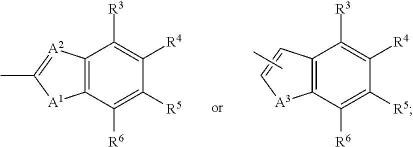

A¹ is NR⁷, O, or S;
A² is N;
A³ is O or S;
R³ through R⁶ are independently hydrogen, halogen, haloalkyl; and
R⁷ is hydrogen, C₁-C₄ alkyl, or C(O)O—(C₁-C₄)-alkyl.
In certain embodiments, R¹ is CO₂R²;
R² is H or tert-butyl;
X¹ is Cl;
X² is Cl;
Y is C═O;
Z is

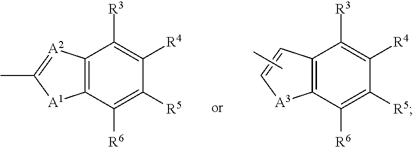

A¹ is NR⁷, O, or S;
A² is N;
A³ is O or S;
R³ through R⁶ are independently hydrogen, halogen, haloalkyl; and
R⁷ is hydrogen, C₁-C₄ alkyl, or C(O)O—(C₁-C₄)-alkyl.
In certain embodiments, R¹ is CO₂R²;
R² is H or tert-butyl;
X¹ is Cl;
X² is Cl;
Y is C═O;
Z is

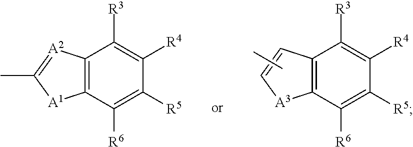

A¹ is NR⁷, O, or S;
A² is N;
A³ is O or S;
R³, R⁵, and R⁶ are hydrogen;
R⁴ is hydrogen, halogen, or haloalkyl; and
R⁷ is hydrogen, C₁-C₄ alkyl, or C(O)O—(C₁-C₄)-alkyl.
In certain embodiments, the compound of Formula (III) is selected from the group consisting of:

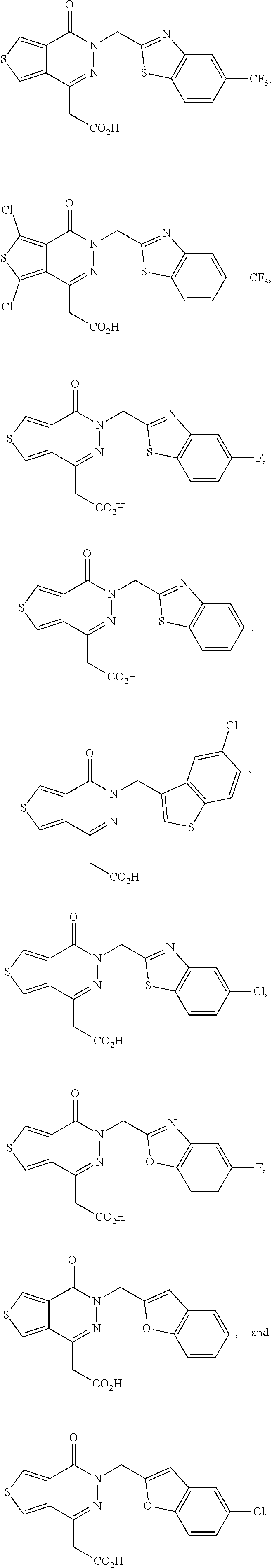

In certain embodiments, the compound of Formula (I) is

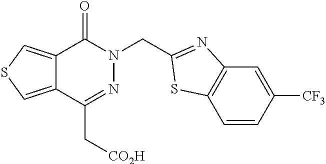 [SMILES: O=C(O)Cc1nn(Cc2nc3cc(C(F)(F)F)ccc3s2)c(=O)c2cscc12]

or a pharmaceutically acceptable salt thereof.

In certain embodiments, the compound of formula (I) is

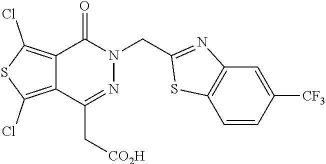 [SMILES: O=C(O)Cc1nn(Cc2nc3cc(C(F)(F)F)ccc3s2)c(=O)c2c(Cl)sc(Cl)c12]

or a pharmaceutically acceptable salt thereof.

### Compounds of Formulas IV, V and VI

The AR inhibitors can be a compound of Formula (IV) or pharmaceutically acceptable salts, and solvates thereof,

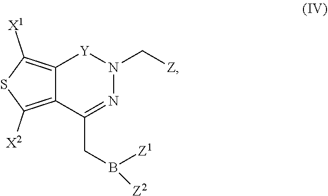

wherein,

X¹ is H or halogen;

X² is H or halogen;

Y is a bond, C═O, C═S, C═NH, or C═N(C₁-C₄)-alkyl;

Z¹ and Z² are independently selected from the group consisting of hydroxy, alkoxy, aryloxy, or Z¹ and Z² taken together with the boron atom to which they are bonded form

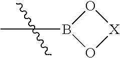

wherein,

X is a substituted or unsubstituted C₂-C₅ alkylene;

Z is

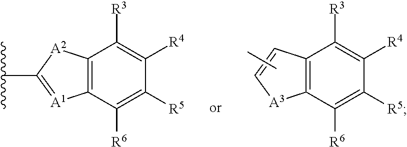

A² is NR⁷, O, S or CH₂;

A¹ is N or CH;

A³ is NR⁷, O, or S;

R³ through R⁶ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl; and

R⁷ is hydrogen, C₁-C₄ alkyl, or C(O)O—(C₁-C₄)-alkyl.

Suitable substituents on the C₂-C₅ alkylene include one or more alkyl, alkoxy, aryl, aryloxy, halo, haloalkyl, haloalkoxy, haloalkylthio. A preferred substituted C₂-C₅ alkylene is substituted ethylene. A more preferred substituted C₂-C₅ alkylene is —C(CH₃)₂C(CH₃)₂—.

It will be recognized by those of skill in the art that the designation of

Z is

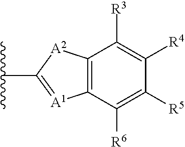

### or Z is

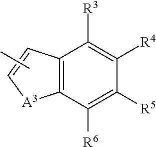

indicates that when Z is

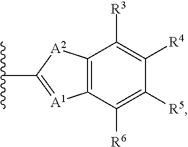

the compounds of Formula (IV) are understood to encompass

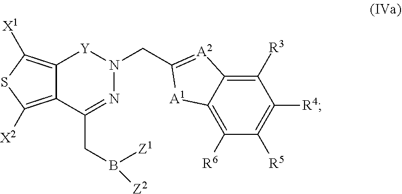

and

when Z is

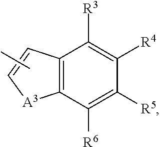

the compounds of Formula (IV) are understood to encompass

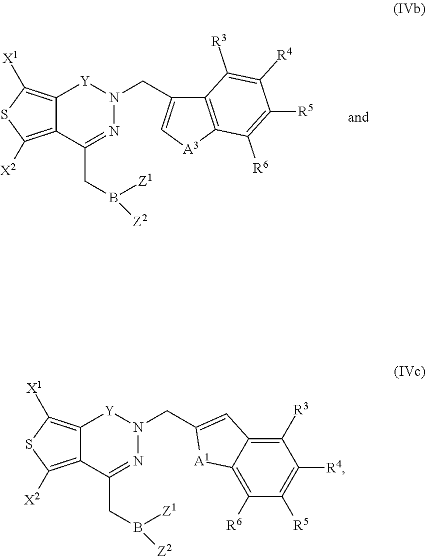

wherein,

Z¹ and Z² are independently selected from the group consisting of hydroxy, alkoxy, aryloxy, or Z¹ and Z² taken together with the boron atom to which they are bonded form

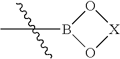

wherein,

X is a substituted or unsubstituted C₂-C₅ alkylene.

In certain embodiments, R³ through R⁶ of Formula (IV) are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl.

In certain embodiments, R³ through R⁶ of Formula (IV) are independently hydrogen, halogen or haloalkyl. In certain embodiments, R³ through R⁶ are independently hydrogen, halogen or trihaloalkyl.

In certain embodiments, R³ and R⁶ of Formula (IV) are hydrogen. In certain embodiments, R³, R⁵, and R⁶ are hydrogen.

In certain embodiments, R⁴ of Formula (IV) is hydrogen, halogen or haloalkyl. In certain embodiments, R⁴ is hydrogen. In certain embodiments, R⁴ is halogen. In certain embodiments, R⁴ is haloalkyl. In certain embodiments, R⁴ is CF₃.

In certain embodiments, R³ through R⁶ of Formula (IV) are hydrogen. In certain embodiments, R³, R⁵, R⁶ are hydrogen and R⁴ is halogen or haloalkyl. In certain embodiments, R³, R⁵, R⁶ are hydrogen and R⁴ is haloalkyl. In certain embodiments, R³, R⁵, R⁶ are hydrogen and R⁴ is CF₃. In certain embodiments, R³, R⁵, R⁶ are hydrogen and R⁴ is halogen. In certain embodiments, R³, R⁵, R⁶ are hydrogen and R⁴ is F. In certain embodiments, R³, R⁵, R⁶ are hydrogen and R⁴ is Cl.

In certain embodiments, Y of Formula (IV) is C═O, C═S, C═NH, or C═N(C₁-C₄)-alkyl. In certain embodiments, Y is C═O or C═S. In certain embodiments, Y is C═O. In certain embodiments, Y is C═S. In certain embodiments, Y is C═NH, or C═N(C₁-C₄)-alkyl.

In certain embodiments, A² of Formula (IV) is NR⁷, O, S or CH₂. In certain embodiments, A² is NR⁷, O, or S. In certain embodiments, A² is NR⁷, S or CH₂. In certain embodiments, A² is NR⁷ or O. In certain embodiments, A² is NR⁷ or S. In certain embodiments, A² is NR⁷. In certain embodiments, A² is O. In certain embodiments, A² is S.

In certain embodiments, A¹ of Formula (IV) is N or CH. In certain embodiments, A¹ is N. In certain embodiments, A¹ is CH.

In certain embodiments, A³ of Formula (IV) is NR⁷, O, or S. In certain embodiments, A³ is O. In certain embodiments, A³ of Formula (IV) is S. In certain embodiments, A³ is NR⁷.

In certain embodiments, X¹ and X² of Formula (IV) are hydrogen.

In certain embodiments, X¹ and X² of Formula (IV) are halogen. In certain embodiments, X¹ and X² are Cl.

In certain embodiments, X¹ and X² of Formula (IV) are independently hydrogen or halogen. In certain embodiments, X¹ is hydrogen and X² is Cl. In certain embodiments, X¹ is Cl and X² is hydrogen.

In certain embodiments, Z of Formula (IV) is

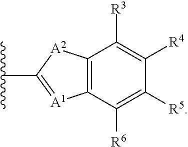

In certain embodiments, Z of Formula (IV) is

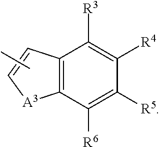

In certain embodiments, R⁷ of Formula (IV) is hydrogen, C₁-C₄ alkyl, or C(O)O—(C₁-C₄)-alkyl. In certain embodiments, R⁷ is hydrogen. In certain embodiments, R⁷ is C₁-C₄ alkyl. In certain embodiments, R⁷ is C₁-C₃ alkyl. In certain embodiments, R⁷ is C₁-C₂ alkyl. In certain embodiments, R⁷ is C₁-C₄ n-alkyl. In certain embodiments, R⁷ is C₁-C₃ n-alkyl. In certain embodiments, R⁷ is C(O)O—(C₁-C₄)-alkyl. In certain embodiments, R⁷ is C(O)O—(C₁-C₃)-alkyl. In certain embodiments, R⁷ is C(O)O—(C₁-C₂)-alkyl. In certain embodiments, R⁷ is C(O)O—(C₁-C₄)-n-alkyl. In certain embodiments, R⁷ is C(O)O—(C₁-C₃)-n-alkyl.

In certain embodiments, the compounds of Formula (IV) is

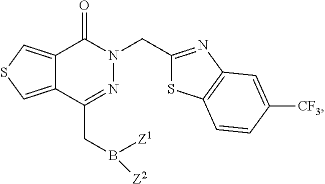

or pharmaceutically acceptable salts, pro-drugs or solvates thereof;

wherein,

Z¹ and Z² are independently selected from the group consisting of hydroxy, alkoxy, aryloxy, or Z¹ and Z² taken together with the boron atom to which they are bonded form

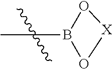

wherein,

X is a substituted or unsubstituted C₂-C₅ alkylene.

In certain embodiments, the compounds of Formula (IV) is

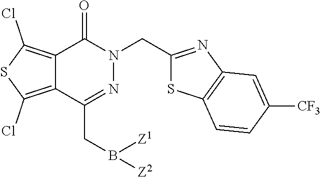

or pharmaceutically acceptable salts, pro-drugs or solvates thereof;

wherein,

Z¹ and Z² are independently selected from the group consisting of hydroxy, alkoxy, aryloxy, or Z and Z² taken together with the boron atom to which they are bonded form

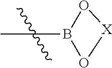

wherein,

X is a substituted or unsubstituted C₂-C₅ alkylene.

In certain embodiments, the compounds of Formula (IV) is

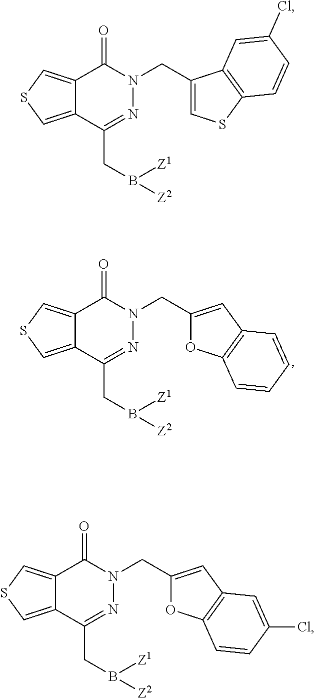

or pharmaceutically acceptable salts, pro-drugs or solvates thereof;

wherein,

Z¹ and Z² are independently selected from the group consisting of hydroxy, alkoxy, aryloxy, or Z¹ and Z² taken together with the boron atom to which they are bonded form

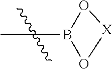

wherein,

X is a substituted or unsubstituted C₂-C₅ alkylene.

In certain embodiments, the compounds of Formula (IV) is

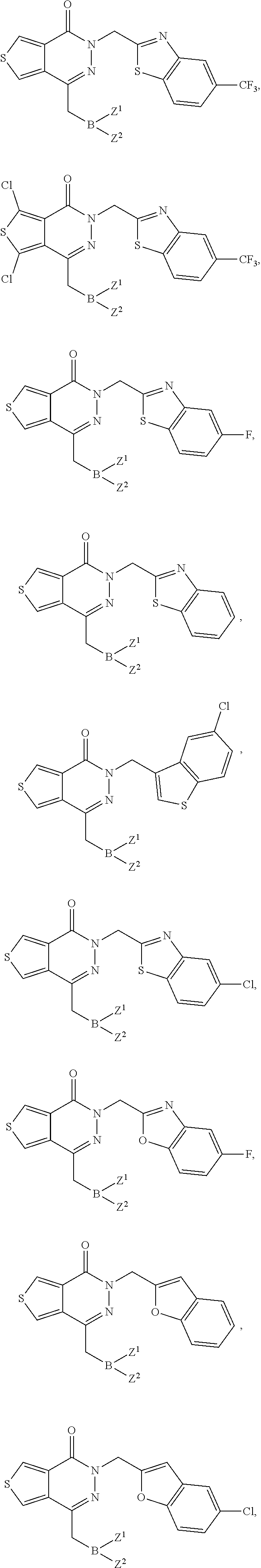

or pharmaceutically acceptable salts, pro-drugs or solvates thereof;

wherein,

Z¹ and Z² are independently selected from the group consisting of hydroxy, alkoxy, aryloxy, or Z¹ and Z² taken together with the boron atom to which they are bonded form

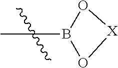

wherein,

X is a substituted or unsubstituted C₂-C₅ alkylene.

In another aspect, the aldose reductase inhibitor is a compound of Formula (V)

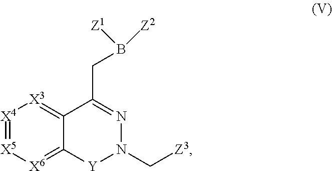

or pharmaceutically acceptable salts, pro-drugs or solvates thereof;

wherein,

X³ is N or CR⁸;

X⁴ is N or CR⁹;

X⁵ is N or CR¹⁰;

X⁶ is N or CR¹¹; with the proviso that two or three of X³, X⁴, X⁵, or X⁶ are N;

Z¹ and Z² are independently selected from the group consisting of hydroxy, alkoxy, aryloxy, or Z¹ and Z² taken together with the boron atom to which they are bonded form

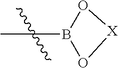

wherein,

X is a substituted or unsubstituted C₂-C₅ alkylene;

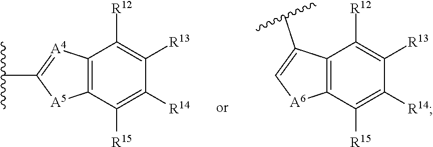

Z³ is

A⁵ is NR¹⁶, O, S or CH₂;

A⁴ is N or CH;

A⁶ is NR¹⁶, O, or S;

R⁸ through R¹⁵ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl; or two of R⁸ through R¹¹ or two of R¹² through R¹⁵ taken together are (C₁-C₄)-alkylenedioxy; and

R¹⁶ is hydrogen, C₁-C₄ alkyl, or C(O)O—(C₁-C₄)-alkyl.

Suitable substituents on the C₂-C₅ alkylene include one or more alkyl, alkoxy, aryl, aryloxy, halo, haloalkyl, haloalkoxy, haloalkylthio. A preferred substituted C₂-C₅ alkylene is substituted ethylene. A more preferred substituted C₂-C₅ alkylene is —C(CH₃)₂C(CH₃)₂—.

It will be recognized by those of skill in the art that the designation of

Z is

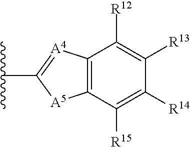

### or Z is

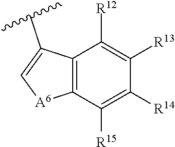

indicates that when Z is

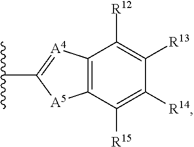

the compounds of Formula (V) are understood to encompass

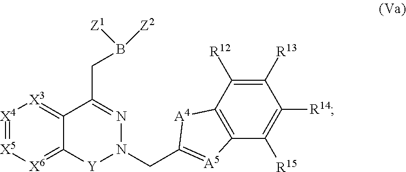

and when Z is

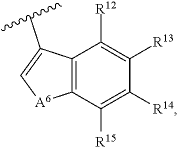

the compounds of Formula (V) are understood to encompass

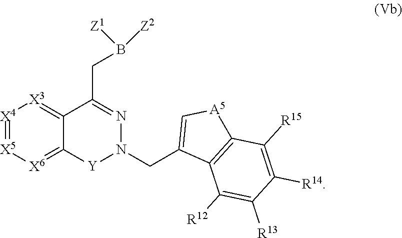

In some compounds of Formula V, R⁸ through R¹⁵ are independently hydrogen, halogen or haloalkyl, for example, R⁸ through R¹⁵ are independently hydrogen, halogen or trihaloalkyl (e.g., —CF₃).

In other compounds of Formula V, R⁸ through R¹¹ are hydrogen.

In certain embodiments of compounds of Formula V, R¹² through R¹⁵ are independently hydrogen, halogen or haloalkyl, for example, R¹² through R¹⁵ are independently hydrogen, halogen or trihaloalkyl (e.g., —CF₃).

In certain embodiments, R¹² and R¹⁵ of Formula (V) are hydrogen.

In certain embodiments, R¹³ of Formula (V) is hydrogen, halogen or haloalkyl. In certain embodiments, R¹³ is hydrogen. In certain embodiments, R¹³ is halogen. In certain embodiments, R¹³ is haloalkyl.

In certain embodiments, R¹⁴ of Formula (V) is hydrogen, halogen or haloalkyl. In certain embodiments, R¹⁴ is hydrogen. In certain embodiments, R¹⁴ is halogen. In certain embodiments, R¹⁴ is haloalkyl.

In certain embodiments, Y of Formula (V) is C═O, C═S, C═NH, or C═N(C₁-C₄)-alkyl. In certain embodiments, Y is C═O or C═S. In certain embodiments, Y is C═O. In certain embodiments, Y is C═S. In certain embodiments, Y is C═NH, or C═N(C₁-C₄)-alkyl.

In certain embodiments, A⁵ of Formula (V) is NR¹⁶, S or CH₂. In certain embodiments, A⁵ is NR¹⁶ or O. In certain embodiments, A⁵ is NR¹⁶ or S. In certain embodiments, A⁵ is NR¹⁶. In certain embodiments, A⁵ is O. In certain embodiments, A⁵ is S.

In certain embodiments, A⁴ of Formula (V) is N or CH. In certain embodiments, A⁴ is N. In certain embodiments, A⁴ is CH.

In certain embodiments, A⁶ of Formula (V) is O or S. In certain embodiments, A⁶ is O. In certain embodiments, A⁶ is S.

In certain embodiments, X³ and X⁶ of Formula (V) are nitrogen.

In certain embodiments, X³ and X⁴ of Formula (V) are nitrogen.

In certain embodiments, X³ and X⁵ of Formula (V) are nitrogen.

In certain embodiments, X⁴ and X⁵ of Formula (V) are nitrogen.

In certain embodiments, X⁴ and X⁶ of Formula (V) are nitrogen.

In certain embodiments, X⁵ and X⁶ of Formula (V) are nitrogen.

In certain embodiments, Z³ of Formula (V) is

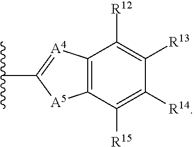

In certain embodiments, Z³ of Formula (V) is

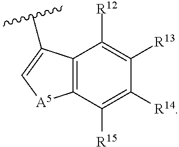

In some embodiments, the compounds of Formula (V) is

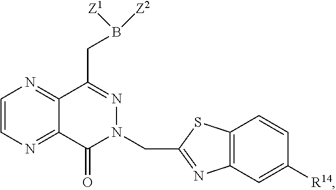

or pharmaceutically acceptable salts, pro-drugs or solvates thereof;

wherein,

R¹⁴ is hydrogen, halogen or trihaloalkyl (e.g., —CF₃); and

Z¹ and Z² are independently selected from the group consisting of hydroxy, alkoxy, aryloxy, or Z¹ and Z² taken together with the boron atom to which they are bonded form

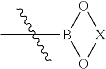

wherein,

X is a substituted or unsubstituted C₂-C₅ alkylene.

In embodiments, the compounds of Formula (V) is

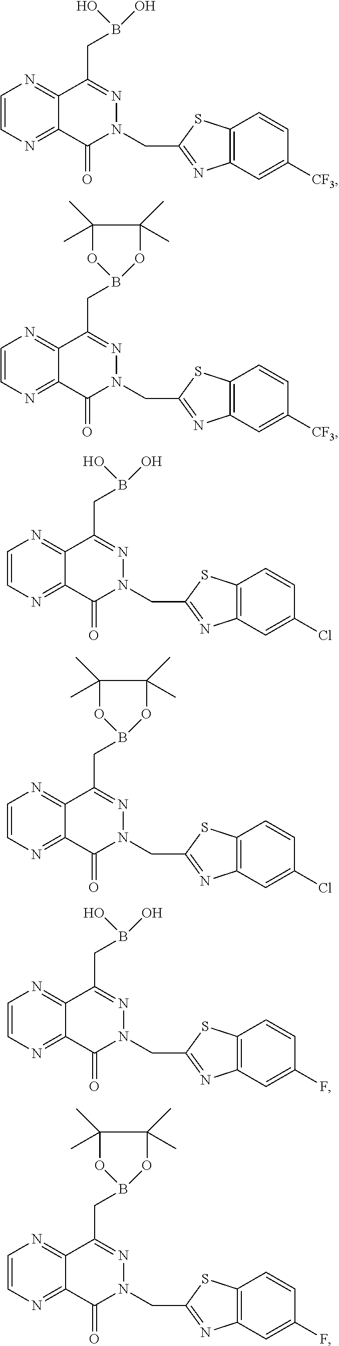

or pharmaceutically acceptable salts, pro-drugs or solvates thereof.

In one aspect, the aldose reductase inhibitor is a compound of Formula (VI)

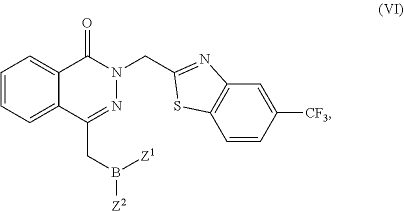

or pharmaceutically acceptable salts, pro-drugs or solvates thereof;

wherein,

Z¹ and Z² are independently selected from the group consisting of hydroxy, alkoxy, aryloxy, or Z¹ and Z² taken together with the boron atom to which they are bonded form

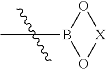

wherein,

X is a substituted or unsubstituted C₂-C₅ alkylene.

In an embodiment, the aldose reductase inhibitor of Formula (VI) is

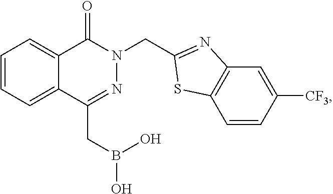 [SMILES: O=c1c2ccccc2c(CB(O)O)nn1Cc1nc2cc(C(F)(F)F)ccc2s1]

or pharmaceutically acceptable salts, pro-drugs or solvates thereof.

In an embodiment, the AH inhibitor of Formula (VI) is

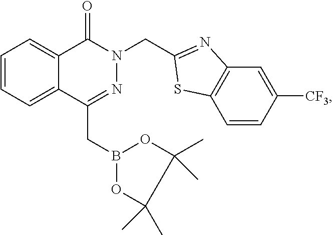 [SMILES: CC1(C)OB(Cc2nn(Cc3nc4cc(C(F)(F)F)ccc4s3)c(=O)c3ccccc23)OC1(C)C]

or pharmaceutically acceptable salts, pro-drugs or solvates thereof.

The term “alkyl”, as used herein, unless otherwise indicated, refers to a monovalent aliphatic hydrocarbon radical having a straight chain, branched chain, monocyclic moiety, or polycyclic moiety or combinations thereof, wherein the radical is optionally substituted at one or more carbons of the straight chain, branched chain, monocyclic moiety, or polycyclic moiety or combinations thereof with one or more substituents at each carbon, where the one or more substituents are independently C₁-C₁₀ alkyl. Examples of “alkyl” groups include methyl, ethyl, propyl, isopropyl, butyl, iso-butyl, sec-butyl, tert-butyl, pentyl, hexyl, heptyl, cyclopropyl, cyclobutyl, cyclopentyl, cyclohexyl, cycloheptyl, norbornyl, and the like.

The term “halogen” or “halo-”, as used herein, means chlorine (Cl), fluorine (F), iodine (I) or bromine (Br).

As used herein, the term “acyl” is used in a broad sense to designate radicals of the type RCO—, in which R represents an organic radical which may be an alkyl, aralkyl, aryl, alicyclic or heterocyclic radical, substituted or unsubstituted, saturated or unsaturated; or, differently defined, the term “acyl” is used to designate broadly the monovalent radicals left when the OH group of the carboxylic radical is removed from the molecule of a carboxylic acid.

The term “alkoxy” is employed to designate a group of the formula: —O—R wherein R is an alkyl group, which optionally contains substituents, such as halogen. Preferably, the term “alkoxy” is employed to designate an alkoxy with an alkyl group of 1 to 6 carbon atoms. Most preferably, the term “alkoxy” is employed to designate an alkoxy with an alkyl group of 1 to 3 carbon atoms, such as methoxy or ethoxy.

The term “cycloalkyl group” is used herein to identify cycloalkyl groups having 3-6 carbon atoms preferably cyclopropyl, cyclobutyl, cyclopentyl and cyclohexyl.

The term “solvate” as used herein means a compound, or a pharmaceutically acceptable salt thereof, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate.”

A “prodrug” refers to an agent, which is converted into the parent drug in vivo. Prodrugs are often useful because, in some situations, they are easier to administer than the parent drug. They are bioavailable, for instance, by oral administration whereas the parent drug is either less bioavailable or not bioavailable. The prodrug also has improved solubility in pharmaceutical compositions over the parent drug. For example, the compound carries protective groups which are split off by hydrolysis in body fluids, e.g., in the bloodstream, thus releasing active compound or is oxidized or reduced in body fluids to release the compound. The term “prodrug” may apply to such functionalities as, for example; the acid functionalities of the compounds of formula I. Prodrugs may be comprised of structures wherein an acid group is masked, for example, as an ester or amide. Further examples of prodrugs are discussed herein. See also Alexander et al. (J. Med. Chem. 1988, 31, 318), which is incorporated by reference. Examples of prodrugs include, but are not limited to, derivatives and metabolites of a compound that include biohydrolyzable moieties such as biohydrolyzable amides, biohydrolyzable esters, biohydrolyzable carbamates, biohydrolyzable carbonates, and biohydrolyzable phosphate analogues. Prodrugs are also described in, for example, The Practice of Medicinal Chemistry (Camille Wermuth, ed., 1999, Academic Press; hereby incorporated by reference in its entirety). In certain embodiments, prodrugs of compounds with carboxyl functional groups are the lower alkyl esters of the carboxylic acid. The carboxylate esters are conveniently formed by esterifying any of the carboxylic acid moieties present on the molecule. Prodrugs can typically be prepared using well-known methods, such as those described by Burger's Medicinal Chemistry and Drug Discovery 6^{th} ed. (Donald J. Abraham ed., 2001, Wiley) and Design and Application of Prodrugs (H. Bundgaard ed., 1985, Harwood Academic Publishers Gmfh; each of which hereby incorporated by reference in its entirety). Biohydrolyzable moieties of a compound of Formula I (a) do not interfere with the biological activity of the compound but can confer upon that compound advantageous properties in vivo, such as uptake, duration of action, or onset of action; or (b) may be biologically inactive but are converted in vivo to the biologically active compound. Examples of biohydrolyzable esters include, but are not limited to, lower alkyl esters, alkoxyacyloxy esters, alkyl acylamino alkyl esters, and choline esters. Examples of biohydrolyzable amides include, but are not limited to, lower alkyl amides, α-amino acid amides, alkoxyacyl amides, and alkylaminoalkylcarbonyl amides. Examples of biohydrolyzable carbamates include, but are not limited to, lower alkylamines, substituted ethylenediamines, amino acids, hydroxyalkylamines, heterocyclic and heteroaromatic amines, and polyether amines.

The term “salt” includes salts derived from any suitable of organic and inorganic counter ions well known in the art and include, by way of example, hydrochloric acid salt or a hydrobromic acid salt or an alkaline or an acidic salt of the aforementioned amino acids. The term is intended to include salts derived from inorganic or organic acids including, for example hydrochloric, hydrobromic, sulfuric, nitric, perchloric, phosphoric, formic, acetic, lactic, maleic, fumaric, succinic, tartaric, glycolic, salicylic, citric, methanesulfonic, benzenesulfonic, benzoic, malonic, trifluoroacetic, trichloroacetic, naphthalene-2 sulfonic and other acids; and salts derived from inorganic or organic bases including, for example sodium, potassium, calcium, ammonium or tetrafluoroborate. Exemplary pharmaceutically acceptable salts are found, for example, in Berge, et al. (J. Pharm. Sci. 1977, 66(1), 1; and U.S. Pat. Nos. 6,570,013 and 4,939,140; each hereby incorporated by reference in its entirety). Pharmaceutically acceptable salts are also intended to encompass hemi-salts, wherein the ratio of compound:acid is respectively 2:1. Exemplary hemi-salts are those salts derived from acids comprising two carboxylic acid groups, such as malic acid, fumaric acid, maleic acid, succinic acid, tartaric acid, glutaric acid, oxalic acid, adipic acid and citric acid. Other exemplary hemi-salts are those salts derived from diprotic mineral acids such as sulfuric acid. Exemplary preferred hemi-salts include, but are not limited to, hemimaleate, hemifumarate, and hemisuccinate.

The term “acid” contemplates all pharmaceutically acceptable inorganic or organic acids. Inorganic acids include mineral acids such as hydrohalic acids, such as hydrobromic and hydrochloric acids, sulfuric acids, phosphoric acids and nitric acids. Organic acids include all pharmaceutically acceptable aliphatic, alicyclic and aromatic carboxylic acids, dicarboxylic acids, tricarboxylic acids, and fatty acids. Preferred acids are straight chain or branched, saturated or unsaturated C₁-C₂₀ aliphatic carboxylic acids, which are optionally substituted by halogen or by hydroxyl groups, or C₆-C₂ aromatic carboxylic acids. Examples of such acids are carbonic acid, formic acid, fumaric acid, acetic acid, propionic acid, isopropionic acid, valeric acid, alpha-hydroxy acids, such as glycolic acid and lactic acid, chloroacetic acid, benzoic acid, methane sulfonic acid, and salicylic acid. Examples of dicarboxylic acids include oxalic acid, malic acid, succinic acid, tartaric acid and maleic acid. An example of a tricarboxylic acid is citric acid. Fatty acids include all pharmaceutically acceptable saturated or unsaturated aliphatic or aromatic carboxylic acids having 4 to 24 carbon atoms. Examples include butyric acid, isobutyric acid, sec-butyric acid, lauric acid, palmitic acid, stearic acid, oleic acid, linoleic acid, linolenic acid, and phenylsteric acid. Other acids include gluconic acid, glycoheptonic acid and lactobionic acid.

### III. Compositions

The compounds can be administered in the form a suitable composition, such as a pharmaceutical composition. Pharmaceutical compositions are physiologically acceptable and typically include the active compound and a carrier. The term “carrier” refers to a diluent, adjuvant, excipient, or vehicle with which a compound is administered. Non-limiting examples of such pharmaceutical carriers include liquids, such as water, alcohols and oils, including those of petroleum, animal, vegetable or synthetic origin, such as peanut oil, soybean oil, mineral oil, sesame oil and the like. The pharmaceutical carriers may also be saline, gum acacia, gelatin, starch paste, talc, keratin, colloidal silica, urea, and the like. In addition, auxiliary, stabilizing, thickening, lubricating and coloring agents may be used. Suitable pharmaceutical carriers, dosage forms and formulation techniques to produce pharmaceutical compositions for a desired type of administration are well-known and are described, for example, in Remington's Pharmaceutical Sciences (Alfonso Gennaro ed., Krieger Publishing Company (1997); Remington's: The Science and Practice of Pharmacy, 21^{st} Ed. (Lippincot, Williams & Wilkins (2005); Modern Pharmaceutics, vol. 121 (Gilbert Banker and Christopher Rhodes, CRC Press (2002); each of which hereby incorporated by reference in its entirety). Suitable pharmaceutical carriers, dosage forms and formulation techniques for dermatological and cosmetic uses are also are well-known and are described, for example, in Handbook of Cosmetic Science and Technology, Fourth Edition, edited by Andre O. Barel, Marc Paye, Howard I. Maibach, CRC Press, 2014, the contents of which is hereby incorporated by reference in its entirety.

The composition can be in a desired form, such as a table, capsule, solution, emulsion, suspension, gel, sol, or colloid that is physiologically and/or pharmaceutically acceptable. Compositions for topical administration or application to the skin are typically in the form of a solution (e.g., water or alcoholic), lotion, cream, emulsion (e.g., oil in water, water in oil), ointment, gel, paste, foam, spray or powder. The compositions can be prepared for administration by any suitable route such as ocular (including periocular and intravitreal administration), oral, parenteral (e.g., subcutaneous, intravenous, intra-arterial, intrathecal and intraperitoneal administration), intranasal, anal, vaginal, and topical administration. Oral or topical administration is generally preferred.

If desired, the pharmaceutical composition can include a buffer, for example with alkaline buffers, e.g., ammonium buffer, acidic buffers, e.g., ethanoates, citrates, lactates, acetates, etc., or zwitterionic buffers, such as, glycine, alanine, valine, leucine, isoleucine and phenylalanine, Kreb's-Ringer buffer, TRIS, MES, ADA, ACES, PIPES, MOPSO, cholamine chloride, MOPS, BES, TES, HEPES, DIPSO, MOBS, TAPSO, acetamidoglycine, TEA, POPSO, HEPPSO, EPS, HEPPS, Tricine, TRIZMA, Glycinamide, Glycyl-glycine, HEPBS, Bicine, TAPS, AMPB, CHES, AMP, AMPSO, CAPSO, CAPS, and CABS.

When the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. If desired, tonicity adjusting agents can be included, such as, for example, sugars, sodium chloride or combinations thereof. In some embodiments, the composition is isotonic.

The compositions may also include additional ingredients, such as acceptable surfactants, co-solvents, emollients, agents to adjust the pH and osmolarity and/or antioxidants to retard oxidation of one or more component.

Suitable carriers for oral administration are well-known and comprise inert diluents, edible carriers or combinations thereof. Examples of pharmaceutically acceptable carriers may include, for example, water or saline solution, polymers such as polyethylene glycol, carbohydrates and derivatives thereof, oils, fatty acids, or alcohols. Surfactants such as, for example, detergents, are also suitable for use in the formulations. Specific examples of surfactants include polyvinylpyrrolidone, polyvinyl alcohols, copolymers of vinyl acetate and of vinylpyrrolidone, polyethylene glycols, benzyl alcohol, mannitol, glycerol, sorbitol or polyoxyethylenated esters of sorbitan; lecithin or sodium carboxymethylcellulose; or acrylic derivatives, such as methacrylates and others, anionic surfactants, such as alkaline stearates, in particular sodium, potassium or ammonium stearate; calcium stearate or triethanolamine stearate; alkyl sulfates, in particular sodium lauryl sulfate and sodium cetyl sulfate; sodium dodecylbenzenesulphonate or sodium dioctyl sulphosuccinate; or fatty acids, in particular those derived from coconut oil, cationic surfactants, such as water-soluble quaternary ammonium salts of formula N R′R″R′″R″″Y″, in which the R radicals are identical or different optionally hydroxylated hydrocarbon radicals and Y″ is an anion of a strong acid, such as halide, sulfate and sulfonate anions; cetyltrimethylammonium bromide is one of the cationic surfactants which can be used, amine salts of formula NR′R′R″, in which the R radicals are identical or different optionally hydroxylated hydrocarbon radicals; octadecylamine hydrochloride is one of the cationic surfactants which can be used, non-ionic surfactants, such as optionally polyoxyethylenated esters of sorbitan, in particular Polysorbate 80, or polyoxyethylenated alkyl ethers; polyethylene glycol stearate, polyoxyethylenated derivatives of castor oil, polyglycerol esters, polyoxyethylenated fatty alcohols, polyoxyethylenated fatty acids or copolymers of ethylene oxide and of propylene oxide, amphoteric surfactants, such as substituted lauryl compounds of betaine.

If desired, an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof, a disintegrating agent, such as, for example, corn starch, potato starch, alginic acid or combinations thereof, a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc., or combinations thereof containing two or more of the foregoing.

Topical formulations for application to the skin typically include one or more of a vehicle or solvent, permeation enhancer, thickening or gelling agent, humectant, emulsifying or solubilizing agent, emollient, stiffening agent or ointment base. See, e.g., Chang et al., The AAPS Journal 15:1, 41-52, 2012. DOI:10.10.1208/s12248-012-9411-0). Suitable vehicles or solvents include, purified water, Hexylene glycol, Propylene glycol, Oleyl alcohol, Propylene carbonate, Mineral oil, and the like. Suitable permeation enhancers include Propylene glycol, Ethanol, Isopropyl Alcohol, Oleic acid, Polyethylene glycol, and the like. Suitable thickening or gelling agents include Carbomer, Methyl cellulose, Sodium carboxyl methyl cellulose, Carrageenan, Colloidal silicon dioxide, Guar gum, Hydroxypropyl cellulose, Hydroxypropyl methyl cellulose, Gelatin, Polyethylene oxide, Alginic acid, Sodium alginate, Fumed silica, and the like. Suitable humectants include Glycerin, Propylene glycol, Polyethylene glycol, Sorbitol solution, 1,2,6 Hexanetriol, and the like. Suitable emulsifying or solubilizing agents include Polysorbate 20, Polysorbate 80, Polysorbate 60, Poloxamer, Emulsifying wax, Sorbitan monostearate, Sorbitan monooleate, Sodium lauryl sulfate, Propylene glycol monostearate, Diethylene glycol monoethyl ether, Docusate sodium, and the like. Suitable emollients, stiffening agents, ointment bases include Carnauba wax, Cetyl alcohol, Cetyl ester wax, Emulsifying wax, Hydrous lanolin, Lanolin, Lanolin alcohols, Microcrystalline wax, Paraffin, Petrolatum, Polyethylene glycol, Stearic acid, Stearyl alcohol, White wax, Yellow wax, and the like. If desired, preservatives, anti-oxidants, chelating agents, acidifying, alkalizing or buffering agents can also be included in topical compositions.

Typical pharmaceutically acceptable compositions can contain a an AR inhibitor and/or a pharmaceutically acceptable salt thereof at a concentration ranging from about 0.01 to about 20 wt %, such as 0.01 to about 15 wt %, 0.01 to about 10 wt %, or about 0.01 to about 5 wt %. The compositions for parenteral administration or typically sterile.

### IV. Combination Therapy

The methods described herein include the administration of an AR inhibitor and one more additional therapeutic agents. The additional therapeutic agents may be administered before, concurrently with or after the AR inhibitor, but in a manner that provides for overlap of the pharmacological activity of the AR inhibitor and the additional therapeutic agent.

The additional therapeutic agent can be, for example, second aldose reductase inhibitor, an antioxidant, or both. For example, the second aldose reductase can zopolrestat, epalrestat, ranirestat, berberine and sorbinil, as described in, e.g., U.S. Pat. Nos. 4,939,140; 6,159,976; and 6,570,013. Preferably, the second aldose reductase inhibitor is selected from ponalrestat, epalrestat, sorbinil or sorbinol, imirestat, AND-138, CT-112, zopolrestat, zenarestat, BAL-AR18, AD-5467, M-79175, tolrestat, alconil, statil, berberine or SPR-210.

Other therapeutic agents that can be administered with an AR inhibitor include, for example, antioxidants and cell regulators. Suitable antioxidants include vitamins, such as vitamin C (L-ascorbic acid), vitamin B₃ (niacinamide) and vitamin E (alpha-tocopheraol), polyphenols (e.g., from green tea) and flavonoids (e.g. from soya). Suitable cell regulators include, for example, retinol and retinol derivatives, such as retinaldehyde and Tretinoin.

### V. Example

Prevention of Skin Cell Damage Caused by Oxidative Stress

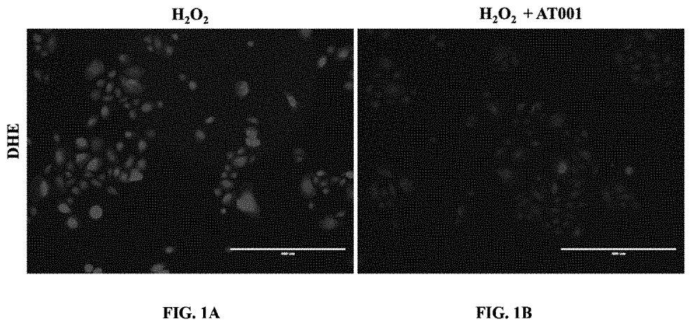

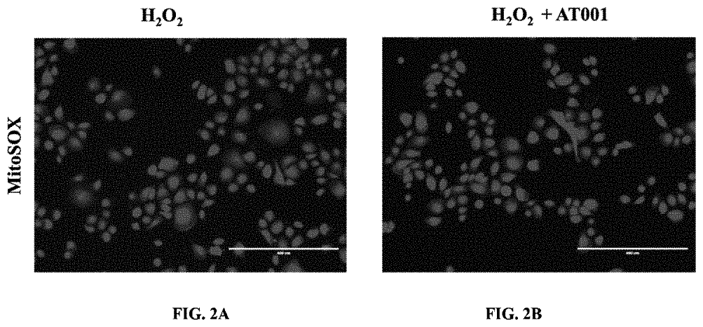

Cultured human skin cells (keratinocytes) were exposed to H₂O₂, which causes oxidative damage to simulate the aging process, in the presence of Compound A (at 0.18 nM concentration) or vehicle control. The cultured cells were then assessed for cytosolic reactive oxygen species via Dihydroethidium (DHE) staining (FIG. 1A and FIG. 1B) and for mitochondrial stress via Mitosox™ (a fluorgenic dye targeted to mitochondria in live cells and readily oxidized by superoxide to produce fluorescence, ThermoFisher Scientific) staining (FIG. 2A and FIG. 2B).

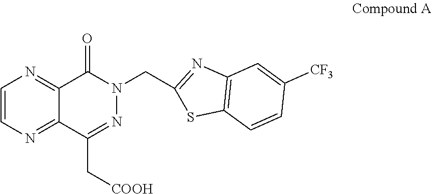 [SMILES: O=C(O)Cc1nn(Cc2nc3cc(C(F)(F)F)ccc3s2)c(=O)c2nccnc12]

Treating the cultured cells with Compound A (at 0.18 nM concentration) attenuated H₂O₂-induced cytosolic reactive oxygen species formation (FIGS. 1A and 1B), and also reduced mitochondrial oxidative stress (FIGS. 2A and 2B). The data demonstrate that aldose reductase inhibitors can protect skin cells from oxidative stress by reducing the level of cytosolic reactive oxygen species and also reducing downstream mitochondrial oxidative stress.

A second aldose reductase inhibitor, Compound B, was tested in the same cultured human keritincyte system. Compound B also inhibited H₂O₂-induced cytosolic reactive oxygen species formation and mitochondrial oxidative stress.

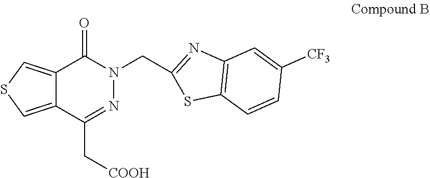 [SMILES: O=C(O)Cc1nn(Cc2nc3cc(C(F)(F)F)ccc3s2)c(=O)c2cscc12]

Senescence-associated beta-galactosidase (SA-βgal) is a well-known biomarker of senescent cells in culture and in vivo. Debacq-Chainiaux et al., Nature Protocols 4, 1798-1806 (2009). In additional studies the cultured human keritinocytes that were exposed to H₂O₂ in the presence of Compound A (at 0.18 nM concentration) or vehicle control were analyzed for expression of SA-βgal using a cytochemical approach. The data demonstrated that Compound A inhibited H₂O₂-induced SA-βgal.

These data validate aldose reductase inhibition as a treatment strategy to prevent skin cell damage due to the aging process.

Unless otherwise defined, all technical and scientific terms used herein have the same meaning as commonly understood by one of ordinary skill in the art to which this disclosure belongs.

Various aspects of the invention have been described in this disclosure. Such aspects may, however, be embodied in many different forms and should not be construed as limited to the particular embodiments set forth herein; rather, these embodiments are provided so that this disclosure will be thorough and complete.

Although methods and materials similar or equivalent to those described herein can be used in the practice or testing of the present disclosure, suitable methods and materials are described in the foregoing paragraphs. In addition, the materials, methods, and examples are illustrative only and not intended to be limiting. All United States patents and published or unpublished United States patent applications cited herein are incorporated by reference. All published foreign patents and patent applications cited herein are hereby incorporated by reference. All published references, documents, manuscripts, scientific literature cited herein are hereby incorporated by reference.

1. A method for treating cutaneous aging, comprising administering to a subject in need thereof a therapeutically effective amount of an aldose reductase inhibitor.

2. The method of claim 1, wherein the method is for reducing or delaying the appearance of lines, creases and/or wrinkles in the skin.

3. The method of claim 1, wherein the aldose reductase inhibitor is topically administered to the skin.

4. The method of claim 3, wherein the aldose reductase inhibitor is topically applied to the surface of the skin.

5. The method of claim 4, wherein the aldose reductase inhibitor is topically applied to the skin of the face, neck, chest, arms, hands or any combination of the foregoing.

6. The method of claim 3, wherein the aldose reductase inhibitor is in the form a topical formulation.

7. The method of claim 1, wherein the therapeutically effective amount is effective to reduce advanced glycation end products (AGEs) in the skin.

8. The method of claim 1, wherein the therapeutically effective amount is effective to reduce reactive oxygen species (ROS) in the skin.

9. The method of claim 1, wherein the therapeutically effective amount is effective to reduce 3-GC in the skin.

10. The method of claim 1, wherein the therapeutically effective amount is effective to reduce fructose in the skin.

11. The method of claim 1, wherein the therapeutically effective amount is effective to reduce oxidative damage in the skin.

12. The method of claim 1, wherein the therapeutically effective amount is effective to reduce or delay fragmentation, break down and/or cross-linking of extracellular matrix proteins in the skin.

13. The method of claim 12, wherein the therapeutically effective amount is effective to reduce fragmentation, break down and/or cross-linking of collagen and/or elastin in the dermis.

14. The method of claim 1, wherein the inhibitor of aldose reductase is administered at least once a day.

15. A method of reducing advanced glycation end products (AGEs), reactive oxygen species (ROS), 3-GC, fructose, or oxidative damage in the skin of a subject, comprising topically administering to the skin of the subject a therapeutically effective amount of an aldose reductase inhibitor.

16-19. (canceled)

20. A method of reducing or delaying fragmentation, break down and/or cross-linking of extracellular matrix proteins in the skin of a subject, comprising topically administering to the skin of the subject a therapeutically effective amount of an aldose reductase inhibitor.

21. The method of claim 15, further comprising systemically administering to the subject that same or different aldose reductase inhibitor.

22. The method of claim 1, wherein the aldose reductase inhibitor is a compound of any one of Formulas I or II 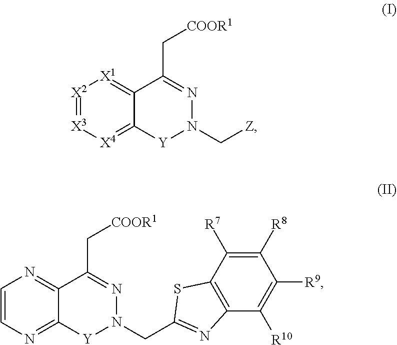
or a salt thereof, wherein
R¹ is H, (C₁-C₆)-alkyl, (C₁-C₆)-hydroxyalkyl, or (C₁-C₆)-aminoalkyl;
X¹ is Nor C R³;
X² is N or CR⁴;
X³ is N or CR⁵;
X⁴ is N or CR⁶; with the proviso that two or three of X¹, X², X³, or X⁴ are N;
Y is a bond, C═O, C═S, C═NH, or C═N(C₁-C₄)-alkyl;
Z is 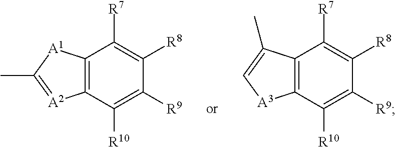
A¹ is NR¹¹, O, S or CH₂;
A² is N or CH;
A³ is NR¹¹, O, or S;
R³ through R¹⁰ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl; or two of R³ through R⁶ or two of R⁷ through R¹⁰ taken together are (C₁-C₄)-alkylenedioxy; and
R¹¹ is hydrogen, C₁-C₄ alkyl, or C(O)O—(C₁-C₄)-alkyl;
or
wherein the aldose reductase inhibitor is a compound of Formula (III): 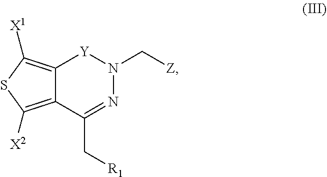
or a salt thereof, wherein:
R¹ is CO₂R² or CO₂⁻X⁺;
R² is H, (C₁-C₆)-alkyl, (C₁-C₆)-hydroxyalkyl, or (C₁-C₆)-aminoalkyl;
X¹ is H or halogen;
X² is H or halogen:
Y is a bond, C═O, C═S, C═NH, or C═N(C₁-C₄)-alkyl;
Z is 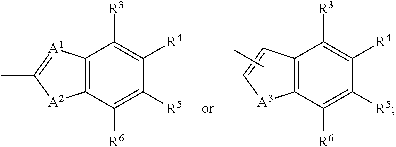
A¹ is NR⁷, O, S or CH₂;
A² is N or CH;
A³ is NR⁷, O, or S;
R³ through R⁶ are independently hydrogen, halogen, cyano, acyl, haloalkyl, haloalkoxy, haloalkylthio, trifluoroacetyl, (C₁-C₄)-alkyl, (C₁-C₄)-alkoxy, (C₁-C₄)-alkylthio, (C₁-C₄)-alkylsulfinyl, or (C₁-C₄)-alkylsulfonyl;
R⁷ is hydrogen, C₁-C₄ alkyl, or C(O)O—(C₁-C₄)-alkyl; and
X⁺ is a counter ion.

23. The method of claim 1, wherein the aldose reductase inhibitor is zopolrestat, epalrestat, or a salt thereof.

24-28. (canceled)
